(19) United States
(12) Patent Application Publication
(10) Pub. No.: US 2007/0244356 A1
(43) Pub. Date: Oct. 18, 2007

(54) Elongate medical devices having an improved distal profile for use with an endoscope

(75) Inventors: Oscar R. Carrillo JR., Attleboro, MA (US); Gary J. Leanna, Holden, MA (US); William J. Shaw, Cambridge, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Boston Scientific Scimed, Inc.

(21) Appl. No.: 11/405,655

(22) Filed: Apr. 17, 2006

(51) Int. Cl. A61B 1/00

(52) U.S. Cl. 600/107; 600/104

(57) ABSTRACT

Endoscopic medical devices and methods for making and using the same. An example medical device for use with an endoscope is a catheter having a key member disposed on the distal end region of the catheter. The key member defines a key region that may be shaped so that at least a portion thereof is complementary or configured to mate with a notch formed in the endoscope elevator.

### FIELD

The present invention pertains to endoscopes and medical devices for use with endoscopes. More particularly, the present disclosure pertains to endoscopic catheters with a key portion or key member disposed thereon.

### BACKGROUND

A wide variety of endoscopes, medical devices for use with endoscopes, and endoscopic procedures have been developed. Of the known endoscopes, medical devices for use with endoscopes, and endoscopic procedures, each has certain advantages and disadvantages. There is an ongoing need to provide alternative medical devices for use with endoscopes as well as methods for making and using medical devices with endoscopes.

### SUMMARY

The invention provides design, material, and manufacturing method alternatives for endoscopes, medical devices for use with endoscopes, and for methods for making and using endoscopes. An example medical device for use with an endoscope is a guidewire, catheter, or any other endoscopic instrument having a key member disposed on its distal end region. The key member defines a key region that may be shaped so that at least a portion thereof is complementary or configured to mate with a notch formed in the endoscope elevator. Some additional details regarding these and other embodiments are described in more detail below.

The above summary of some embodiments is not intended to describe each disclosed embodiment or every implementation of the present disclosure. The Figures and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of an illustrative endoscopic instrument assembly;

FIG. 1A is a top view of the endoscopic instrument assembly shown in FIG. 1;

FIG. 1B is a partial cross-sectional side view of the endoscopic instrument assembly shown in FIG. 1 where the elevator is down;

FIG. 1C is a partial cross-sectional side view of the endoscopic instrument assembly shown in FIG. 1 where the elevator is up;

FIG. 2 is a perspective view of an illustrative key member disposed on a shaft;

FIG. 3 is a perspective view of another illustrative key member disposed on a shaft;

FIG. 4 is a perspective view of another illustrative key member disposed on a shaft;

FIG. 5 is a perspective view of the illustrative key member depicted in FIG. 4 engaged with the elevator of an endoscope;

FIG. 6 is a perspective view of an illustrative key member disposed on a shaft;

FIG. 7 is a perspective view of the illustrative key member depicted in FIG. 6 engaged with the elevator of an endoscope;

FIG. 8 is a perspective view of another illustrative key member disposed on a shaft;

FIG. 8A is a perspective view of an alternative embodiment of the key member depicted in FIG. 8;

FIG. 9 is a perspective view of the illustrative key member depicted in FIG. 8 engaged with the elevator of an endoscope;

FIG. 10 is a perspective view of an illustrative guidewire having a key region;

FIG. 11 is a perspective view of another illustrative guidewire having a key region; and

FIG. 12 is a perspective view of another illustrative key member disposed on a shaft.

### DETAILED DESCRIPTION

The following description should be read with reference to the drawings wherein like reference numerals indicate like elements throughout the several views. The detailed description and drawings, which are not necessarily to scale, illustrate example embodiments of the claimed invention and are not intended to limit the scope of the invention.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

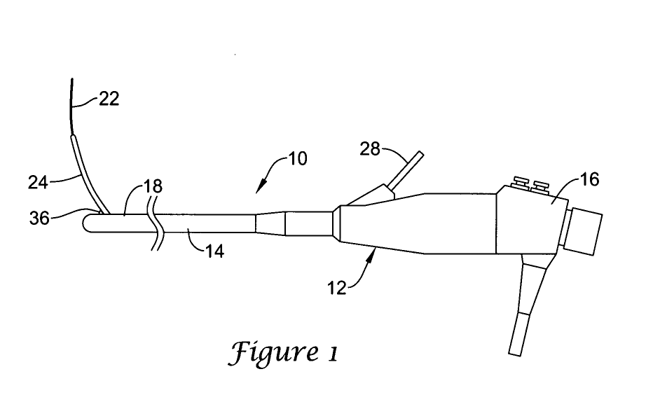

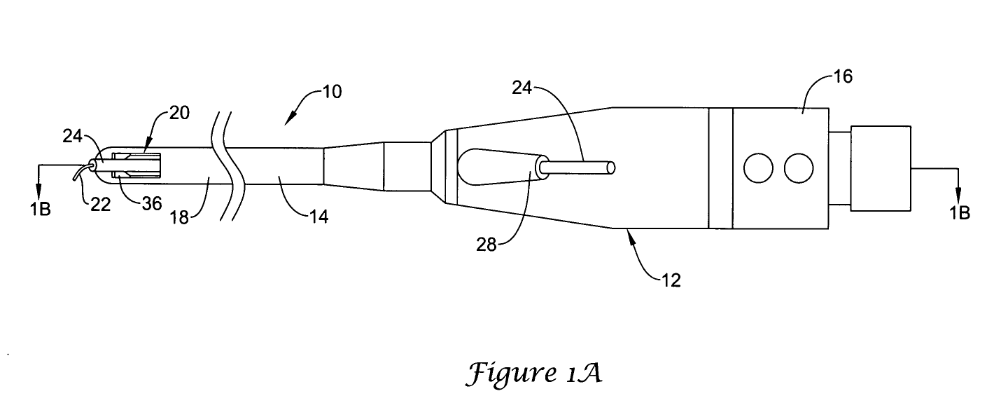

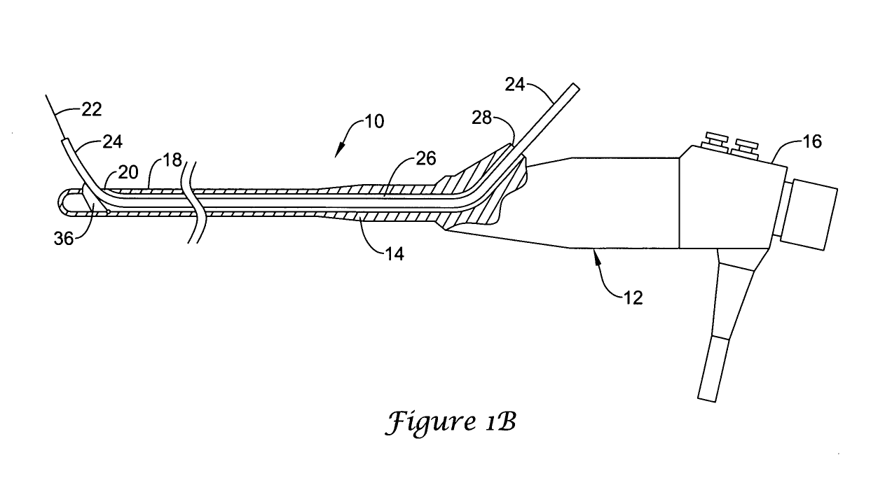

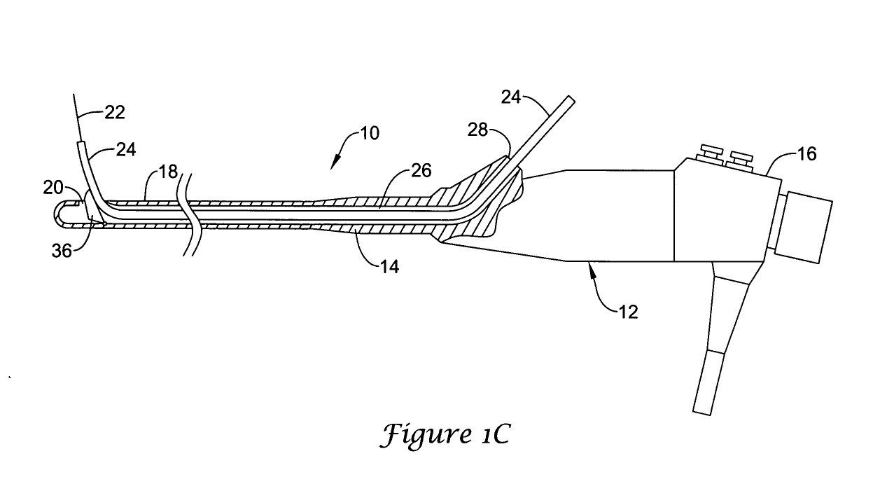

FIG. 1 depicts an example endoscopic instrument assembly 10. Assembly 10 includes an endoscope 12 having a shaft portion 14 and a handle portion 16. Shaft portion 14 includes a distal end region 18 and a distal port 20, where one or more medical devices (e.g., a guidewire 22 and/or a catheter 24) disposed within a working channel 26 (not shown, best seen in FIGS. 1B-1C) formed in shaft portion 14 can extend through. An elevator 36 can be disposed adjacent port 20 (not shown, best seen in FIGS. 1A-1C) that, when actuated, alters the angle at which guidewire 22 and/or catheter 24 exits port 20. Handle portion 16 includes one or more openings or a control region 28 where instruments (e.g., endoscopic instruments, guidewires, catheters, and the like) can gain access to working channel 26 and can be extended through shaft portion 14 and out from port 20. Control region 28 may also include a control wire (not shown), a control dial (not shown), both, or any other suitable means for controlling elevator 36.

FIGS. 1A-1C depict alternative views of assembly 10 in order to illustrate other features thereof. For example, FIG. 1A depicts a top view of assembly 10 where port 20 can be more clearly seen. Similarly, FIGS. 1B-1C depict partial cross-sectional side views of assembly 10 where working channel 26 can be more clearly seen. Moreover, FIG. 1B depicts elevator 36 in the “down” position, whereas FIG. 1C depicts elevator 36 in the “up” position.

During at least some interventions, the position of the endoscopic device and/or the position of a particular medical device disposed in the working channel of the endoscopic device is important. This is at least partially due to challenges in precisely navigating the endoscope or endoscopic instruments through the anatomy of a patient. For example, when endoscopes are used for biliary applications, it may be difficult to advance an endoscope through the papilla of Vater and toward the bile duct. Moreover, once a device is successfully advanced through the papilla of Vater, subtle movement of the devices can result in the device being withdrawn back out from the papilla of Vater, necessitating another round of skilled maneuvering in order to proceed with the intervention.

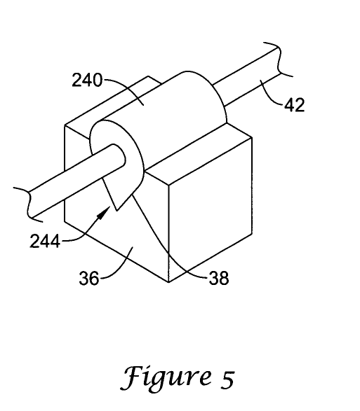

Referring now also to FIG. 5, because of this potential complication, a number of endoscopes, including endoscope 12, include a notch 38 formed in the endoscope elevator 36 that helps to improve the ability of a user to secure the position of a medical device (e.g., guidewire 22, catheter 24, etc.) within shaft portion 14. In order to secure the position of guidewire 22 and/or catheter 24 within shaft portion 14, a user may raise or otherwise actuate elevator 36 (e.g., by actuating the control wire) so that elevator 36 presses and holds guidewire 22 and/or catheter 24 against the wall of shaft portion 14. Notch 38, by virtue of its shape, leaves an opening for guidewire 22 and/or catheter 24 to be disposed in and yet be held in place.

The shape of notch 38 can vary in a number of embodiments. At least some of the contemplated shapes are disclosed herein. For example, notch 38 may have a “V” or “U” shape. Alternatively, the shape of notch 38 can be rounded, oval, non-symmetrical, irregular, polygonal, or resemble any other shape. Regardless of what shape notch 38 has, the relationship between the shape of notch 38 and the shape of a given medical device (e.g., guidewire 22 and/or catheter 24) extending through the working channel of the endoscope 12 may impact the ability of a given endoscope to hold the position of the medical device. Because catheters and guidewires typically have a generally round cross-sectional shape, the shape of these devices may be less than fully complementary with the shape of a V- or U-shaped notch 38. It may be desirable to form a mating or complementary relationship to exist between the shape of the device and the shape of notch 38.

FIG. 2 illustrates an example key member or adapter 40 disposed on the surface of an example medical device 42 that has the ability to alter the shape relationship between notch 38 and device 42. As depicted in FIG. 2, the cross-sectional shape of device 42 is round like a number of typical catheters and guidewires. Key member 40, however, has an alternative, non-circular shape and defines a shaped key region 44 that is designed to be complementary with and/or be configured to mate with notch 38. For example, key member 40 may have a triangular cross-sectional shape so that a pie-shaped or pointed key region 44, corresponding to one “arm” of the triangle, is defined that is complementary with a V- or similarly-shaped notch 38. It can be appreciated that the other “arms” of the triangle-shaped key member 40 also define a suitable key region 44 that is analogously complementary to the shape of notch 38. The complementary shapes of key region 44 and notch 38 may impact the ability of medical device 44 to be secured by elevator 36.

It should be noted that medical device 42 as shown is depicted generically due to the fact that device 42 may take the form of any suitable medical device. For example, in some embodiments, medical device 42 may be an endoscopic guide catheter. However, catheter 42 need not necessarily be a guide catheter as catheter 42 can be any suitable catheter, guidewire, or related medical device for use with endoscopes. The use of catheter 42 may be similar to the use of typical endoscopic catheters. For example, catheter 42 may be advanced through the working channel of an endoscope to a location adjacent an area of interest. Catheter 42 may then be used for its intended purpose. For example, if catheter 42 is a guide catheter, then another diagnostic or therapeutic medical device may be advanced over or through (i.e., through a lumen defined therein) catheter 42.

Key member 40 can be disposed at any position along the length of device 42. For example, key member 40 may be disposed at or near a distal end region of device 42. Alternatively, key member 40 may be disposed at any other suitable location. In addition, key member 40 may span a portion or all of the length of device 42. In some of these as well as other embodiments, key member 40 may be formed in or be part of a sheath or other component disposed along the exterior of device 42. The sheath may be similar to those commonly disposed along the exterior of a number of different medical devices.

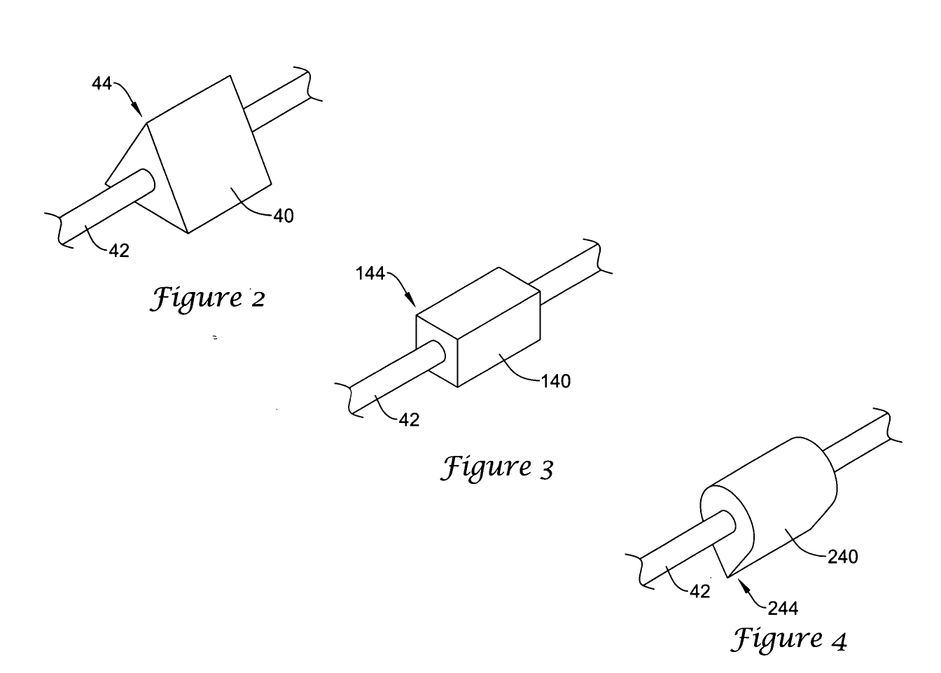

One can readily appreciate that many alternatives can be utilized for notch 38 and/or key region 44 without departing from the spirit of the invention. For example, FIG. 3 depicts key member 140 that has a square cross-sectional shape. Just like in the triangle-shaped version, square key member 140 similarly defines key region 144 that is complementary to a “V” or similarly shape notch 38. Additionally, FIG. 4 depicts another example key member 240 that has a tear drop cross-sectional shape and defines key region 244. FIG. 5 depicts that complementary or mating arrangement of notch 38 with key region 244. Because of the tear-drop shape of key member 240, only a singular key region 244 is defined. Because only a singular key region 244 is defined, the user can track or otherwise determine the rotational orientation of device 42 by simply engaging key region 244 with notch 38. Numerous other embodiments of key members are contemplated that can analogously be used to keep track of orientation.

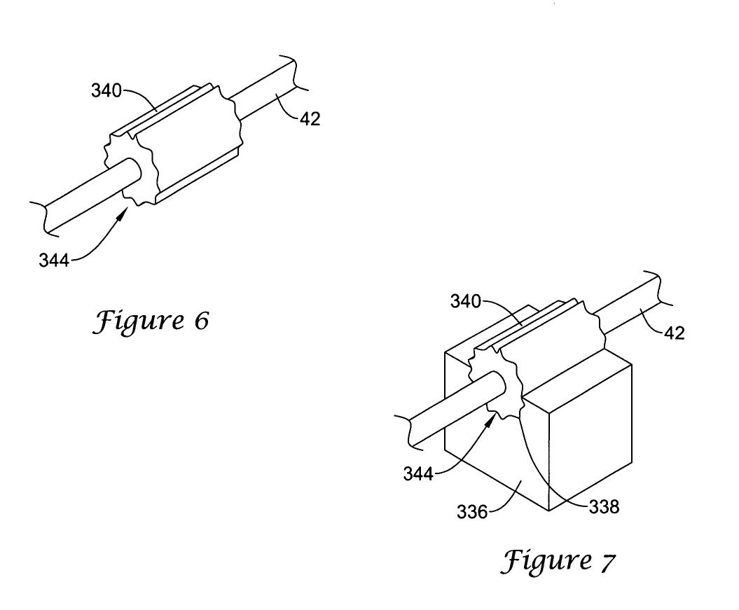

By no means is the shape of notch 38 intended to be limited to just V-shaped or is the shape of the various disclosed key region 44/144/244 intended to be complementary or mate with only V-shaped notches. Any suitable pair of complementary shapes can be utilized. For example, FIG. 6 depicts key member 340 having a cross-sectional shape resembling the teeth on a gear. This key member 340 defines key region 344 that can mate with a complementary notch 338 formed in elevator 336 as shown in FIG. 7. This design theme of utilizing a pair of complementary shapes can be utilized to create these and countless other contemplated mating pairs of key regions and notches.

As alluded to above, the aforementioned key members 40/140/240/340 can also be described as adapters that can be slid onto or disposed about the outer surface of medical device 42 in order to provide device 42 with a key region 44/144/244/344 having the desired shape. The adapters 40/140/240/340 can be removed from device 42 by sliding off (in at least some embodiments) or by cutting it off, for example. Removing adapter 40/140/240/340 from device 42 allows the user to replace one adapter 40/140/240/340 with another, which may be differently shaped, if desired.

The adapters 40/140/240/340 can be made from any suitable material such as a polymer. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In at least some embodiments, the polymeric adapter can be made from a suitable stiff polymer so that a predicable amount of stiffness can be added to medical device 42. In addition (or alternatively) the outer surface of the adapter may be textured so that adapter 40/140/240/340 may frictionally engage notch 38/338. These design considerations may be utilized to impact the integrity of the bond between key regions 44/144/244/344 and notch 38/338 by providing a less resilient surface for the bond interaction to take place.

Alternatively, the polymeric adapter can have a flexibility that resembles or is more flexible than device 42. In some embodiments, these “flexible” embodiments of adapters 40/140/240/340 may resemble a fluid or gel sack (e.g., having a gelatin or silicon-like consistency) that can conform to the shape of any notch 38. These embodiments allow a single version of an adapter 40/140/240/340 to be used with any notch 36 and, thus, any endoscope.

In addition, some of the contemplated adapters 40/140/240/340 can be doped with or otherwise include a radiopaque material. Radiopaque materials are understood to be materials capable of producing a relatively bright image on a fluoroscopy screen or another imaging technique during a medical procedure. This relatively bright image aids the user of device 42 in determining and/or monitoring its location. Some examples of radiopaque materials can include, but are not limited to, gold, platinum, molybdenum, palladium, tantalum, tungsten or tungsten alloy, and the like.

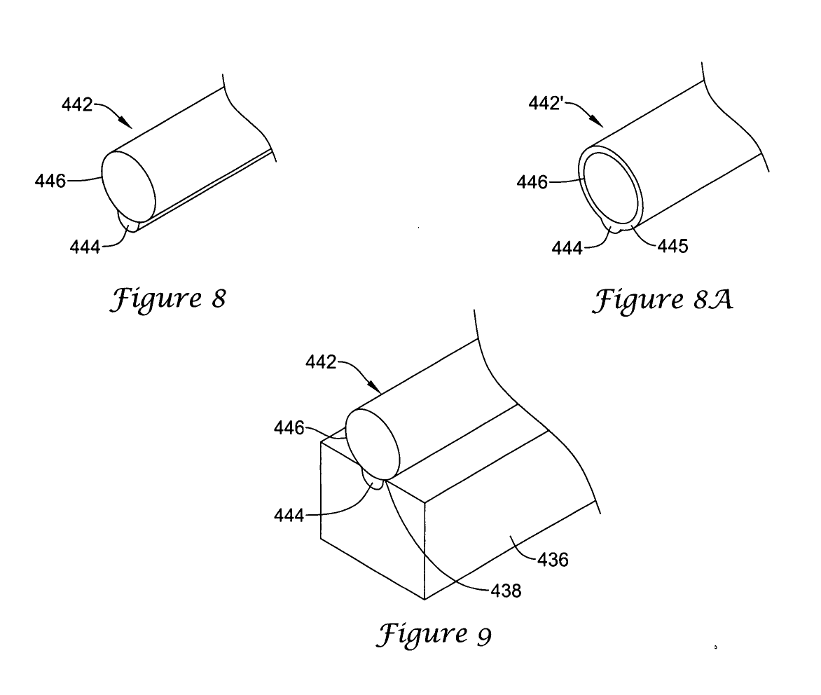

FIG. 8 illustrates another embodiment of a key region 444. Key region 444 is similar to the other disclosed key regions except that rather than taking the form of an adapter, key region 444 is defined by a strip of material disposed on the exterior wall surface 446 of medical device 442. Alternatively, key member 444 may comprise a portion of an exterior jacket or sheath 445 disposed on exterior wall surface 446 of device 442′ as depicted in FIG. 8A. Sheath 445 may be made of a polymeric material such as any of those listed herein or any other suitable material. It should be noted that, to the extent applicable, discussion relating to device 442 can be attributed to device 442′ and vice versa. Likewise, a sheath similar to sheath 445 may be utilized with any of the other shafts/devices disclosed herein to achieve the desired result.

Just like device 42, medical device 442 is depicted generically and may represent any suitable medical device. Key region 444 may extend along only a portion of the device 442 (e.g., the distal end region) or it may extend along essentially the full length of device 442. In at least some embodiments, the strip of material may be a polymeric strip that can be extruded onto or adhesively bonded with device 442. Alternatively, the strip of material may be a metal band that is welded onto, crimped onto, mechanically attached to, or otherwise disposed on device 442.

Key region 444 may be designed to have a shape similar to any of the other key regions disclosed above or contemplated. As shown in FIG. 8, key region 44 may have a partially rounded cross-sectional shape so that it can mate with a U-shaped notch 438 in elevator 436 as shown in FIG. 9.

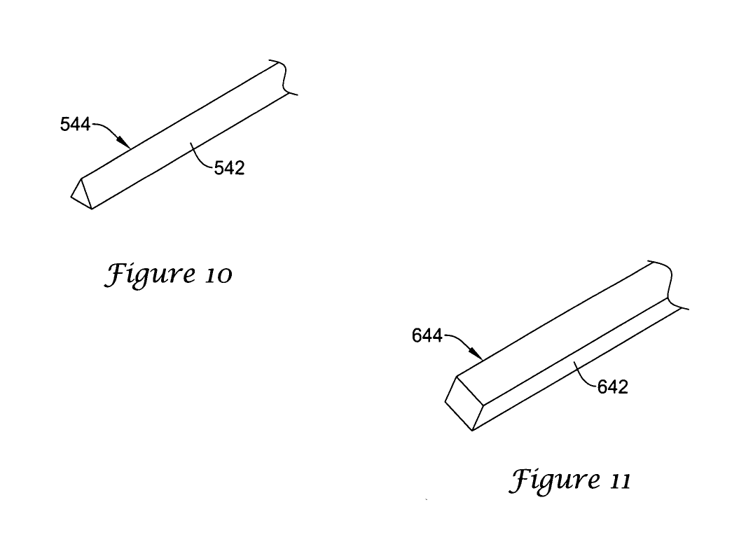

FIGS. 10 and 11 illustrate that forming a keying, mating, or complementary relationship may also be desirable for locking the position of a guidewire in endoscope 12. For example, FIG. 10 illustrates an example guidewire 542 that has a key region 544 with triangular cross-sectional shape. The shape of guidewire 542 and key region 544 may allow guidewire 542 to be secured in position by elevator 36 having a “V” or similarly shaped notch 36. Likewise, FIG. 11 illustrates guidewire 642 having a key region 644 with a square cross-sectional shape that can be similarly secured. Key region 544/644 may span either a portion or the full length of guidewires 542/642.

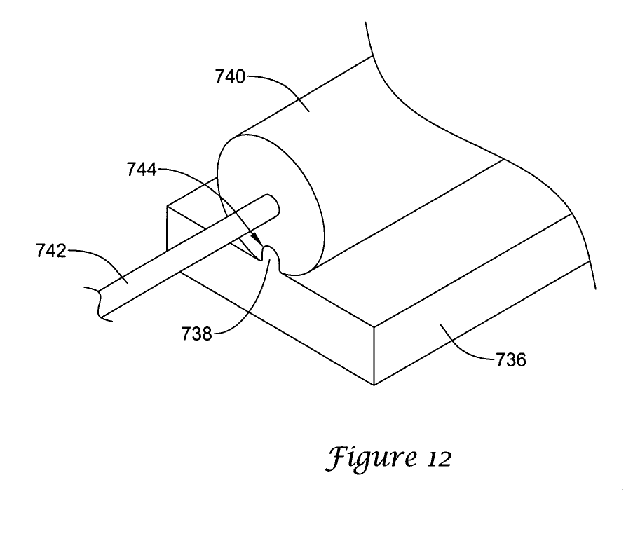

In the foregoing discussion, the various key regions have taken the form of a radially outward or “male” projection disposed on the medical device and the various elevators have included complimentary “female” notches. While numerous embodiments are contemplated with this type of arrangement, other embodiments are contemplated with the reverse arrangement. For example, FIG. 12 illustrates another example device 742 with key member 740 disposed thereon. Key member 740 includes key region 744, which takes the form of a “female” notch. Elevator 736 has a complimentary “male” projection 738. The complimentary relationship between projection 738 and key region 744 allows device 742 to be desirably held in position as well as benefit from the desirable characteristics of this arrangement. It can be appreciated that similar “reverse” arrangements are contemplated for the other embodiments shown herein as well as those others within the scope of the invention.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An endoscopic instrument assembly, comprising:
an endoscope having a handle portion, a shaft portion, a distal port formed in the shaft portion, and a working channel formed in the shaft portion;
an elevator disposed adjacent the distal port, the elevator including a notch having a shape; and
an elongate shaft disposed in the working channel, the shaft having a distal end region;
wherein, the shaft includes a key region along at least the distal end region, the key region having a shape that is at least in part complementary to the shape of the notch.

2. The endoscopic instrument assembly of claim 1, wherein the key region includes a strip of material disposed on the distal end region of the shaft.

3. The endoscopic instrument assembly of claim 1, wherein the key region is defined by an adapter disposed on the distal end region of the shaft.

4. The endoscopic instrument assembly of claim 1, wherein the key region has a triangular cross-sectional shape.

5. The endoscopic instrument assembly of claim 1, wherein the key region has a tear-drop cross-sectional shape.

6. The endoscopic instrument assembly of claim 1, wherein the key region has a square cross-sectional shape.

7. The endoscopic instrument assembly of claim 1, wherein the key region has a non-symmetrical cross-sectional shape.

8. The endoscopic instrument assembly of claim 1, wherein a portion of the key region has a cross-sectional shape that is complementary to the shape of the notch.

9. The endoscopic instrument assembly of claim 1, wherein the notch is substantially V-shaped.

10. The endoscopic instrument assembly of claim 1, wherein the notch is substantially U-shaped.

11. The endoscopic instrument assembly of claim 1, wherein the shaft is a catheter shaft.

12. The endoscopic instrument of claim 1, wherein the shaft is a guidewire.

13. The endoscopic instrument of claim 1, wherein the key region extends along a portion of the length of the shaft.

14. The endoscopic instrument of claim 1, wherein the key region extends along the entire length of the shaft.

15. The endoscopic instrument of claim 1, wherein the key region is defined along at least a portion of a sheath disposed on the shaft.

16. The endoscopic instrument of claim 1, wherein the key region comprises a projection extending radially outward from the shaft and wherein the notch comprises an inward deflection formed in the elevator.

17. The endoscopic instrument of claim 1, wherein the key region comprises an inward deflection formed in the shaft and wherein the notch comprises a projection extending radially outward from the elevator.

18. An endoscopic instrument assembly, comprising:
an endoscope having a handle portion, a shaft portion, a distal port formed in the shaft portion, and a working channel formed in the shaft portion;
an elevator disposed adjacent the distal port, the elevator including a notch having a shape; and
a catheter disposed in the working channel, the catheter having a distal end region;
wherein, the catheter includes a key member disposed on the distal end region that has a shape that is complementary to the shape of the notch.

19. The endoscopic instrument assembly of claim 18, wherein the key member includes a strip of material disposed on the distal end region of the catheter.

20. The endoscopic instrument assembly of claim 18, wherein the key member is defined by an adapter disposed on the distal end region of the catheter.

21. The endoscopic instrument assembly of claim 20, wherein the adapter has a triangular cross-sectional shape.

22. The endoscopic instrument assembly of claim 20, wherein the adapter has a tear-drop cross-sectional shape.

23. The endoscopic instrument assembly of claim 20, wherein the adapter has a square cross-sectional shape.

24. The endoscopic instrument assembly of claim 20, wherein the adapter has a non-symmetrical cross-sectional shape.

25. The endoscopic instrument assembly of claim 20, wherein a portion of the adapter has a cross-sectional shape that is complementary to the shape of the notch.

26. The endoscopic instrument assembly of claim 18, wherein the key member is defined along at least a portion of a sheath disposed on the catheter.

27. The endoscopic instrument assembly of claim 18, wherein the key member comprises a projection extending radially outward from the catheter and wherein the notch comprises an inward deflection formed in the elevator.

28. The endoscopic instrument assembly of claim 18, wherein the key member comprises an inward deflection formed in the catheter and wherein the notch comprises a projection extending radially outward from the elevator.

29. An endoscopic instrument assembly, comprising:
an endoscope having a handle portion, a shaft portion, a distal port formed in the shaft portion, and a working channel formed in the shaft portion;
an elevator disposed adjacent the distal port, the elevator including a notch having a shape; and
a catheter disposed in the working channel, the catheter having a distal end region;
wherein, the catheter includes a key member that is configured to mate with the notch.
